(19) United States
(12) Patent Application Publication
(10) Pub. No.: US 2016/0361513 A1
(43) Pub. Date: Dec. 15, 2016

(54) ELECTRONIC DEVICE WITH HUMIDIFYING MODULE

(71) Applicants: HONG FU JIN PRECISION INDUSTRY (WUHAN) CO., LTD., Wuhan (CN); HON HAI PRECISION INDUSTRY CO., LTD., New Taipei (TW)

(72) Inventor: DONG-BO WANG, Wuhan (CN)

(21) Appl. No.: 14/750,744

(22) Filed: Jun. 25, 2015

(30) Foreign Application Priority Data
Jun. 9, 2015 (CN) 201510310803.1

(51) Int. Cl. A61M 16/16; A61M 16/10

(52) U.S. Cl. CPC A61M 16/16; A61M 16/109; A61M 2205/3368; A61M 2205/586; A61M 2205/8262

(57) ABSTRACT

An electronic device in which or by which air humidity can be controlled includes an enclosure and a humidifying module. The humidifying module is located in the enclosure. The humidifying module includes a chamber and a launcher. The launcher is movable relative to the chamber. The chamber stores the fluid required to create humidity. When the launcher is open, the humidification fluid in the form of vapor can emanate from the fluid chamber.

### CROSS-REFERENCE TO RELATED APPLICATIONS

This application claims priority to Chinese Patent Application No. 201510310803.1 filed on Jun. 9, 2015, the contents of which are incorporated by reference herein.

### FIELD

The subject matter herein generally relates to atmospheric controls for electronic devices.

### BACKGROUND

A humidifier is a device that increases humidity in office or home. However, when the humidifier is in use, the humidifier needs to be particularly placed and be connected to a power supply.

### BRIEF DESCRIPTION OF THE DRAWINGS

Implementations of the present technology will now be described, by way of example only, with reference to the attached figures.

FIG. 1 is a schematic view of an electronic device in a first embodiment.

FIG. 2 is a cross sectional view of the electronic device along line II-II of FIG. 1.

FIG. 3 is a partial view of the humidifying module of FIG. 2, the launcher in an open state.

FIG. 4 is a partial view of a humidifying module in a second embodiment.

FIG. 5 is similar to FIG. 4, the launcher in an open state.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series and the like.

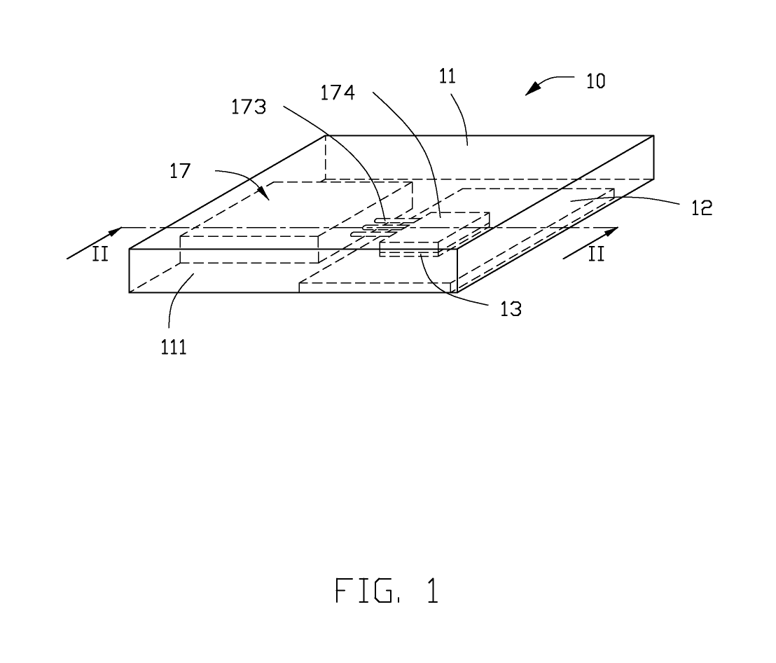

FIG. 1 illustrates an electronic device in a first embodiment. The electronic device 10 includes an enclosure 11, a motherboard 12, a heat element 13, and a humidifying module 17. The humidifying module 17 is placed in the enclosure 11. The enclosure 11 includes a side wall 111. An outer side of the humidifying module 17 is substantially flush with the side wall 111.

The electronic device 10 can be a computer case, a tablet computer, a laptop computer, or a cell phone.

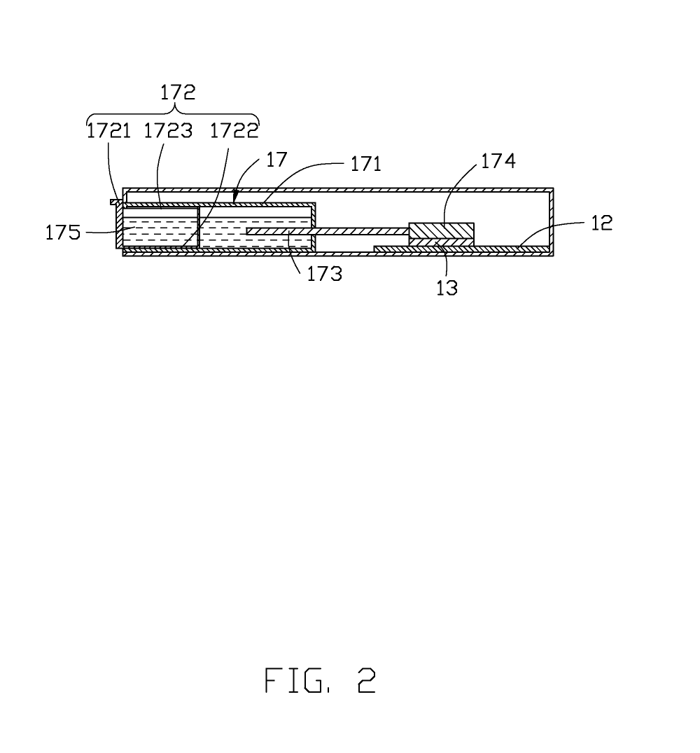

FIG. 2 illustrates a cross section view of the humidifying module 17 along line II-II in the first embodiment. The humidifying module 17 can include a chamber 171, a launcher 172, a plurality of heat pipes 173, and a heat sink 174. The chamber 17 can include heat conducting material. The chamber 171 can be a container to store humidification fluid 175. The humidification fluid 175 can be water. The heat sink 174 contacts the heat element 13. The number of heat pipes 173 in the plurality can be three. Each heat pipe 173 connects to the heat sink 174 and can extend into the chamber 171 from outside of the chamber 171. Each heat pipe 173 can contact the humidification fluid 175. Heat can be conducted from the heat element 13 to the humidification fluid 175 and the chamber 171 through the heat sink 174 and the heat pipes 173.

The launcher 172 can include a baffle 1721, a body 1722, and a vapour outlet plate 1723. The baffle 1721 is placed on an outer side of the chamber 171. The body 1722 can store the humidification fluid 175. The body 1722 can receive the humidification fluid 175 at a side away from the baffle 1721. The vapour outlet plate 1723 is horizontally placed. A level of the vapour outlet plate 1723 is higher than a fluid level of the humidification fluid 175. The vapour outlet plate 1723 can define a plurality of holes or slots from which vapour can emanate.

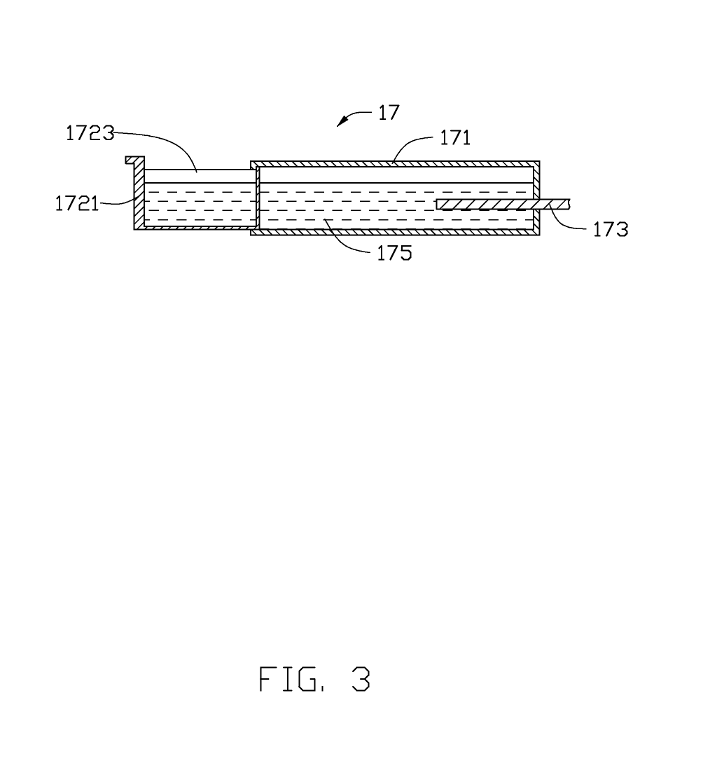

FIG. 3 illustrates the humidifying module 17 in the first embodiment. When the electronic device is working, the launcher 175 can be drawn out of the chamber 171 along a horizontal direction. Heat can be conducted from the heat element into the humidification fluid 175. The humidification fluid 175 is heated to vaporize through the vapour outlet plate 1723.

The humidifying module 17 can further include a detector for monitoring the fluid level inside the humidifying module 175. The humidifying module 175 in the chamber 171 can be topped up via the vapour outlet plate 1723. Rubber can seal the launcher 175 with the chamber 171.

The side wall 1721 of the chamber 171 is moveable between a closed position and an open position. When the side wall 1721 is in the closed position the chamber 171 is sealed. When the side wall 1721 is in the open position, the chamber 171 expands in size to define an expanded area that is open to the enclosure. Fluid within the chamber 171 flows into the expanded area. Heat can vaporize fluid within the chamber 171 than escapes into the enclosure when the side wall 1721 is in the open position.

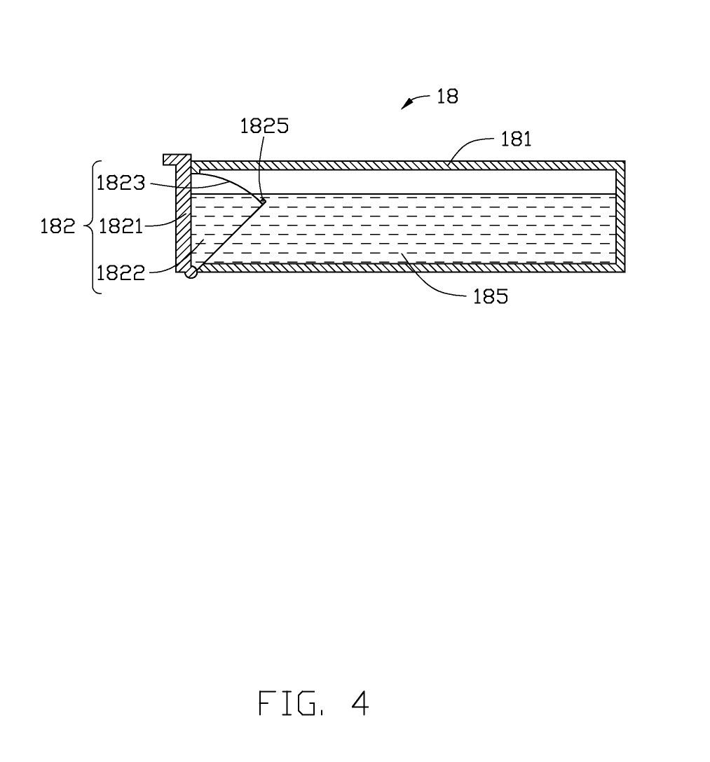

FIG. 4 illustrates a humidifying module 18 in a second embodiment. The humidifying module 18 can include a chamber 181 and a launcher 182. The launcher 182 can include a baffle 1821, a body 1822, and a vapour outlet plate 1823. The baffle 1821 is placed on an outer side of the chamber 181. The body 1822 can store the humidification fluid 185. The launcher 182 is rotatable relative to the chamber 181. A cross section of the vapour outlet plate 1823 is substantially arcuate. A block 1825 is located on the vapour outlet plate 1823 to restrict rotation.

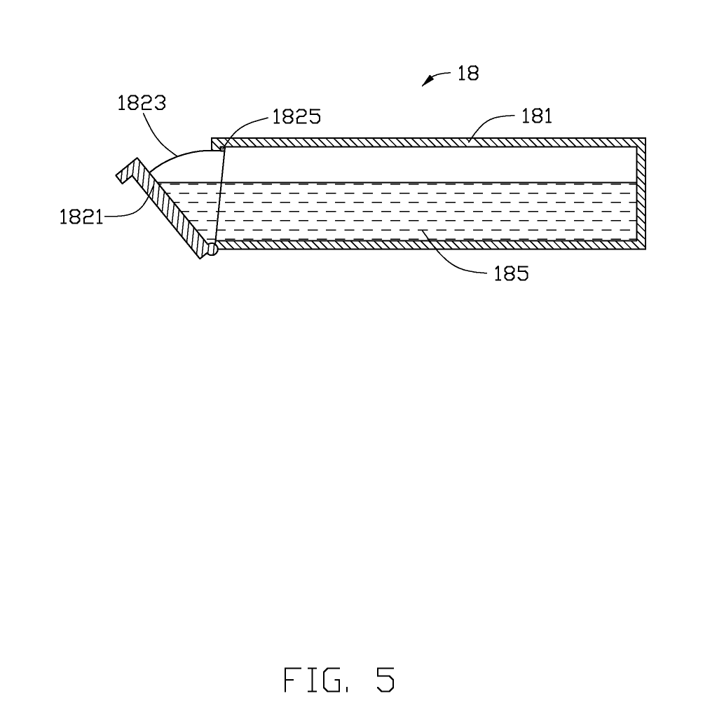

FIG. 5 illustrates the humidifying module 18 in the second embodiment. When an electronic device is working, the launcher 185 can be rotated out of the chamber 181. The humidification fluid 175 is heated to vaporize through the vapour outlet plate 1823.

The embodiments shown and described above are only examples. Many details are often found in the art such as the other features of an electronic device. Therefore, many such details are neither shown nor described. Even though numerous characteristics and advantages of the present technology have been set forth in the foregoing description, together with details of the structure and function of the present disclosure, the disclosure is illustrative only, and changes can be made in the details, including in matters of shape, size, and arrangement of the parts within the principles of the present disclosure, up to and including the full extent established by the broad general meaning of the terms used in the claims. It will therefore be appreciated that the embodiments described above can be modified within the scope of the claims.

What is claimed is:

1. An electronic device comprising:
an enclosure; and
a humidifying module located in the enclosure, and comprising a chamber and a launcher, the launcher being movable relative to the chamber, and the chamber storing humidification fluid,
wherein when the launcher is open, the humidification fluid is configured to spray out of the water chamber for humidifying.

2. The electronic device of claim 1, further comprising a heat element, wherein the humidifying module thermally contacts the heat element.

3. The electronic device of claim 2, wherein the humidifying module further comprising a heat pipe, and the heat pipe is thermally connected to the chamber and the heat element.

4. The electronic device of claim 3, wherein the heat pipe extends into an inner side of the chamber and contacts the humidification fluid.

5. The electronic device of claim 1, wherein the humidification fluid is water, and the humidification fluid is evaporated.

6. The electronic device of claim 1, wherein the launcher is configured to be drawn out of the chamber.

7. The electronic device of claim 6, wherein the launcher further comprises a baffle and a vapour outlet plate, the baffle is placed on an outer side of the chamber, and a level of the vapour outlet plate is higher than a fluid level of the humidification fluid, when the launcher is moved out of the chamber, the humidification fluid is configured to vaporize from the vapour outlet plate.

8. The electronic device of claim 1, wherein the launcher is rotatable relative to the chamber.

9. The electronic device of claim 8, wherein the launcher further comprises a baffle and a vapour outlet plate, the baffle is placed on an outer side of the chamber, when the launcher is rotated out of the chamber, the humidification fluid is configured to vaporize from the vapour outlet plate.

10. The electronic device of claim 9, wherein a cross section of the vapour outlet plate is substantially arcuate.

11. An electronic device comprising:
an enclosure;
a heat element located in the enclosure; and
a humidifying module located in the enclosure and thermally contacts the heat element, and comprising a chamber and a launcher, the launcher being movable relative to the chamber, and the chamber storing humidification fluid,
wherein when the launcher is open, the humidification fluid is configured to be heated by the heat element and be vaporized out of the water chamber.

12. The electronic device of claim 11, wherein the humidifying module further comprising a heat pipe, and the heat pipe is thermally connected to the chamber and the heat element.

13. The electronic device of claim 12, wherein the heat pipe extends into an inner side of the chamber and contacts the humidification fluid.

14. The electronic device of claim 11, wherein the launcher is configured to be drawn out of the chamber.

15. The electronic device of claim 14, wherein the launcher further comprises a baffle and a vapour outlet plate, the baffle is placed on an outer side of the chamber.

16. The electronic device of claim 15, wherein the vapour outlet plate is horizontally placed, and a level of the vapour outlet plate is higher than a fluid level of the humidification fluid, when the launcher is moved out of the chamber, the humidification fluid is configured to vaporize from the vapour outlet plate.

17. The electronic device of claim 11, wherein the launcher is rotatable relative to the chamber.

18. The electronic device of claim 17, wherein the launcher further comprises a baffle and a vapour outlet plate, the baffle is placed on an outer side of the chamber, when the launcher is rotated out of the chamber, the humidification fluid is configured to vaporize from the vapour outlet plate.

19. The electronic device of claim 18, wherein a cross section of the vapour outlet plate is substantially arcuate.

20. A humidifier for an electronic device cover, comprising:
an enclosure;
a fluid container in the enclosure, the fluid container having a side wall moveable between a closed position and an open position; and
a heater configured to heat the fluid container;
wherein when the side wall is in the closed position the fluid container is sealed;
wherein when the side wall is in the open position, the fluid container expands in size to define an expanded area that is open to the enclosure, and fluid within the fluid container flows into the expanded area; and
wherein heat from the heater vaporizes fluid within the fluid container than escapes into the enclosure when the side wall is in the open position.
